(19) United States
(12) Patent Application Publication
(10) Pub. No.: US 2025/0281440 A1
(43) Pub. Date: Sep. 11, 2025

(54) METHOD TO IMPROVE GROWTH PERFORMANCE, LIVABILITY, CARCASS CHARACTERISTICS AND/OR MILK QUALITY IN SWINE

(71) Applicant: NutriQuest, LLC, Mason City, IA (US)

(72) Inventors: Ran Song, Mason City, IA (US); Kim G. Friesen, Mason City, IA (US); Nick W. Shelton, Mason City, IA (US)

(21) Appl. No.: 19/072,645

(22) Filed: Mar. 6, 2025

Related U.S. Application Data
(60) Provisional application No. 63/562,061, filed on Mar. 6, 2024.

(51) Int. Cl. A61K 31/198; A23K 20/142; A23K 50/30; A61P 1/14

(52) U.S. Cl. CPC A61K 31/198; A23K 20/142; A23K 50/30; A61P 1/14

(57) ABSTRACT

The present disclosure provides feed compositions comprising a basal animal diet supplemented with dimethylglycine. The feed compositions improve growth performance, livability, carcass characteristics, and milk quality in non-human animals fed the feed compositions.

### CROSS-REFERENCE TO RELATED APPLICATIONS

This application claims the benefit of U.S. Provisional Application 63/562,061, filed on Mar. 6, 2024, which is hereby incorporated by reference in its entirety.

### FIELD OF THE INVENTION

The present disclosure generally relates to a feed additive composition. More particularly, the present disclosure is concerned with a feed additive composition comprising a glycine compound. The present disclosure also relates to the use of such composition to improve characteristics in swine across multiple parities.

### BACKGROUND OF THE INVENTION

Dimethylglycine (DMG) is a derivative of the amino acid glycine, and it plays a crucial role in various metabolic processes within the body. Structurally, DMG is composed of two methyl groups (—CH₃) attached to a glycine molecule. Therefore, in terms of glycine equivalence, one molecule of DMG is equivalent to one molecule of glycine, with the addition of two methyl groups.

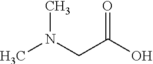

DMG plays a pivotal role in the one-carbon metabolism pathway, critical for synthesizing vital molecules like DNA, proteins, neurotransmitters, and phospholipids. Acting as a methyl donor, DMG contributes its methyl group to molecules such as homocysteine, facilitating the production of methionine, an essential amino acid. This mechanism aids in regulating homocysteine homeostasis, which is crucial for preventing associated health complications, including bone disorders.

In humans, elevated homocysteine (hyperhomocysteinemia) is associated with osteoarthritis and osteoporosis and can cause detrimental effects in bone matrix (Vacek et al., 2013). In rats, DMG supplementation significantly reduced plasma homocysteine level when folate was sufficient (Liu et al., 2021). The connections between hyperhomocysteinemia and bone disorders and ability of DMG to control circulating homocysteine concentration facilitate our invention of using DMG supplementation to prevent pig morbidity caused by lameness and joint disorders via preventing hyperhomocysteinemia, especially in high-lean pigs. Moreover, the demethylation of DMG yields glycine, an amino acid pivotal for muscle development, creatine synthesis, and the production of glutathione—an indispensable antioxidant.

Energy availability is vital for supporting lean tissue growth in pigs through enhancing protein synthesis, cell proliferation, tissue repair, and metabolic regulation. The energy requirement for growing animals is influenced by various factors such as age, sex, genotype, physiological state, and environmental conditions (Baldwin, 1995). Notably, Noblet et al. (1999) observed that high lean-genotype pigs with greater muscle mass exhibited higher maintenance energy requirements compared to those with less muscle mass. Particularly during periods of rapid growth and increased energy demand, strategies aimed at enhancing energy availability to fast-growing animals are crucial to realizing their full potential for lean tissue development. In pigs fed rations not limited in amino acids, the performance response to increasing levels of energy intake depends on the corresponding change in protein and lipid tissue accretion rates in addition to level of intake that corresponds to stage of production. For example, following weaning and, the pig cannot consume sufficient dietary energy (at an economical cost) to full achieve the maximal protein accretion rate. This is commonly referred to as the pig being in an energy dependent phase of growth. As pigs mature, their energy intake increases exponentially and eventually reaches a point in which they consume adequate energy to fully achieve the maximal amount of protein accretion and then additional energy intake is devoted to increased lipid accretion. When they reach that point, they are considered in a protein dependent phase of growth. At the same time, there is a threshold in which protein accretion plateaus and then will begins to drop off. This is the driving factor for why feed conversion becomes much worse in late finishing as the pigs are decreasing the amount of protein deposition and lipid accretion is increasing rapidly which requires more energy intake per unit deposited than protein deposition.

International Applications WO 2007/107184 and WO 2009/033502 disclose the use of DMG in an amount of between 0.001 and 0.5% by dry weight of feed for lowering the feed conversion ratio and increasing the body weight gain of respectively broilers and pigs. Cools et al., (2010) and Kalmar et al., (2010) reported improved nutrient digestibility in sows fed DMG from day 105 of gestation until day 3 of lactation, and in broilers, respectively. Notably, changes of loin muscle deposition, milk quality, and incidence of lameness as a result of supplementation of DMG were not reported in any prior art. Taken the above-mentioned modes of actions together, this invention aims to provide a novel method to enhance lean tissue growth and deposition in pig carcasses, improve overall milk quality, and incidence of lameness through DMG supplementation.

### BRIEF DESCRIPTION OF THE FIGURES

The following drawings form part of the present specification and are included to further demonstrate certain embodiments of the present disclosure. Certain embodiments can be better understood by reference to one or more of these drawings in combination with the detailed description of specific embodiments presented herein.

FIG. 1 is a plot showing loin depth in carcasses of pigs fed DMG (78%) at 0, 0.25, 0.50 and 0.75 lb/ton.

FIG. 2 is a plot showing 10th-rib backfat depth in carcasses of pigs fed DMG (78%) at 0, 0.25, 0.50 and 0.75 lb/ton.

FIG. 3 is a plot showing fat-free carcass lean of pigs fed DMG (78%) at 0, 0.25, 0.50 and 0.75 lb/ton.

FIG. 4 is a plot showing non-esterified fatty acids in the serum of lactating sows fed the control diet without any added DMG or a diet with DMG (78%) at 0.50 lb/ton.

### DETAILED DESCRIPTION

The present disclosure is based on the discovery that administering a feed composition comprising a glycine compound to animals improves characteristics across multiple parities.

The glycine compound includes but is not limited to N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, trimethylglycine, or mixtures or salts or esters thereof.

In some embodiments, the glycine compound may be DMG.

Additional embodiments and features are set forth in part in the description that follows, and in part will become apparent to those skilled in the art upon examination of the specification or may be learned by the practice of the embodiments discussed herein.

A further understanding of the nature and advantages of certain embodiments may be realized by reference to the remaining portions of the specification the drawings, the chemical structures, and descriptions, which forms a part of this disclosure. Any description of any R-group or chemical substituent, alone or in any combination, may be used in any chemical Formula described herein, and Formulae include all conformational and stereoisomers, including diastereomers, epimers, and enantiomers. Moreover, any feature of a composition disclosed herein may be used in combination with any other feature of a composition disclosed herein.

### I. Feed Composition

One aspect of the present disclosure encompasses feed compositions for non-human animals. The non-human animal may be a ruminant or a non-ruminant animal. Examples of ruminant animals include but are not limited to cattle, wild bovines, goats, sheep, giraffes, deer, gazelles, or antelopes. Examples of non-ruminant animals include but are not limited to swine, poultry, horses, dogs, or cats.

In particular, non-human animals may be a swine. The swine may be boars or sows, barrows or gilts. The swine may also be finishing pigs.

The compositions comprise a basal animal diet supplemented with a glycine compound, which may be DMG. The incorporation of a glycine compound has surprisingly been found to be extremely effective in improving milk quality in sows and more generally growth performance, livability, and/or carcass characteristics in commercial finishing pigs.

The carcass characteristics include but are not limited to measurements of muscle and fat deposition. Examples of characteristics measured include but are not limited to loin depth, loin eye area, fat depth, carcass yield, and/or lean percentage.

In some aspects, the feed composition may be used for improving carcass characteristics in a non-human animal.

In some aspects, the feed composition may be used for improving milk composition in a non-human animal. The milk composition comprises milk fat content, milk energy concentration, and/or milk protein content.

In some aspects, the feed composition may be used for increasing protein deposition and reducing protein turnover in a non-human animal.

In some aspects, the feed composition may be used for improving milk production in a non-human animal.

In some aspects, the feed composition may be used for improving growth performance in a non-human animal in a non-human animal.

In some aspects, the feed composition may be used for improving livability in a non-human animal in a non-human animal.

In some aspects, the feed composition may be used for reducing morbidity in a non-human animal. Some examples of causes of morbidity include but are not limited to lameness, abdominal rupture, bloat, genetic deformities, poor intake, injury, prolapse, intestinal torsion, tail bite, and the like.

In some aspects, the feed composition may be used for reducing greenhouse gas emissions and carbon footprint.

### A. Glycine Compounds—Dimethylglycine

The glycine compound in the feed may be N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, trimethylglycine, or mixtures or salts or esters thereof.

In some embodiments, the glycine compound may be DMG.

The amount of DMG in a feed composition can and will vary depending on the body weight, age, and medical condition of the non-human animal and can be determined experimentally. Generally, the amount of DMG present in the formulation will be at an effective amount to improve characteristics in the non-human animal. The term “effective amount” describes an amount of DMG present in a feed composition sufficient to produce a noticeable effect, for example the improved quality of milk production by the subject sow, as determined according to observations described herein. The effective amount will depend on factors such as individual animal parameters including age, physical condition, size and weight; concurrent treatments; the frequency of treatment; or the mode of administration. These factors are well known to those of ordinary skill in the art.

The amount of DMG present in the feed may be at a concentration of about 0.1 lb DMG/ton feed to about 2.0 lb DMG/ton feed. For example, the amount may be from about 0.2 lb DMG/ton feed to about 1.9 lb DMG/ton feed, about 0.3 lb DMG/ton feed to about 1.8 lb DMG/ton feed, about 0.4 lb DMG/ton feed to about 1.7 lb DMG/ton feed, about 0.5 lb DMG/ton feed to about 1.6 lb DMG/ton feed, about 0.6 lb DMG/ton feed to about 1.5 lb DMG/ton feed, about 0.7 lb DMG/ton feed to about 1.4 lb DMG/ton feed, about 0.8 lb DMG/ton feed to about 1.3 lb DMG/ton feed, about 0.9 lb DMG/ton feed to about 1.2 lb DMG/ton feed, or about 1.0 lb DMG/ton feed to about 1.1 lb DMG/ton feed. In some embodiments, the amount of DMG in the feed may be from about 0.25 lb/ton to about 0.5 lb/ton.

Stating in another way, the percent of DMG by dry weight of feed may be from about 0.005% to about 0.1%. For example, the percent of DMG by dry weight of feed may be from about 0.01% to about 0.09%, about 0.02% to about 0.08%, about 0.03% to about 0.07%, or about 0.04% to about 0.06%.

The amount of DMG present in the feed may be at a concentration of about 0.05 g DMG/kg feed to about 1.0 g DMG/kg feed. For example, the amount may be from about 0.06 g DMG/kg feed to about 0.9 g DMG/kg feed, about 0.07 g DMG/kg feed to about 0.8 g DMG/kg feed, about 0.08 g DMG/kg feed to about 0.7 g DMG/kg feed, about 0.09 g DMG/kg feed to about 0.6 kg DMG/kg feed, about 0.1 g DMG/kg feed to about 0.5 kg DMG/kg feed, or about 0.2 g DMG/kg feed to about 0.4 kg DMG/kg feed.

b. Basal Animal Diet

A basal animal diet suitable for a feed composition of the disclosure can and will vary depending on the intended animal, the weight of the animal, and the stage of development of the animal among other variables.

The terms “feed”, “food”, and “feed formulation” are used herein interchangeably and may refer to any feed composition normally fed to an animal. Basal animal diets normally fed to an animal are known in the art. A basal animal diet may include one or more components of an animal feed. Non-limiting examples of feed matter or animal feed matter may include, without limitation: corn or a component of corn, such as, for example, corn meal, corn fiber, corn hulls, corn DDGS (distiller's dried grain with solubles), silage, ground corn, corn germ, corn gluten, corn oil, or any other portion of a corn plant; soy or a component of soy, such as, for example, soy oil, soy meal, soy hulls, soy silage, ground soy, or any other portion of a soy plant; wheat or any component of wheat, such as, for example, wheat meal, wheat fiber, wheat hulls, wheat chaff, ground wheat, wheat germ, or any other portion of a wheat plant; rice or any component of rice, such as, for example, rice meal, rice fiber, rice hulls, rice chaff, ground rice, rice germ, or any other portion of a rice plant; canola, such as, for example, canola oil, canola meal, canola protein, canola hulls, ground canola, or any other portion of a canola plant; sunflower or a component of a sunflower plant; sorghum or a component of a sorghum plant; sugar beet or a component of a sugar beet plant; cane sugar or a component of a sugarcane plant; barley or a component of a barley plant; palm oil, palm kernel or a component of a palm plant; glycerol; corn steep liquor; a waste stream from an agricultural processing facility; lecithin; rumen protected fats; molasses; soy molasses; flax; peanuts; peas; oats; grasses, such as orchard grass and fescue; fish meal, meat & bone meal; feather meal; and poultry byproduct meal; and alfalfa and/or clover used for silage or hay, and various combinations of any of the feed ingredients set forth herein, or other feed ingredients generally known in the art.

As it will be recognized in the art, a basal animal diet may further be supplemented with amino acids, vitamins, minerals, and other feed additives such as other types of enzymes, organic acids, essential oils, probiotics, prebiotics, antioxidants, pigments, anti-caking agents, and the like, as described further below. A basal animal diet may be formulated for administration to any animal subject. Animal subjects may be as described below.

The basal animal diets may optionally comprise at least one additional nutritive and/or pharmaceutical agent. For instance, the at least one additional nutritive and/or pharmaceutical agent may be selected from the group consisting of vitamin, mineral, amino acid, antioxidant, probiotic, essential fatty acid, and pharmaceutically acceptable excipient. The compositions may include one additional nutritive and/or pharmaceutical component or a combination of any of the foregoing additional components in varying amounts. Suitable examples of each additional component are detailed below.

i. vitamins

Optionally, the animal feed formulation may include one or more vitamins. Suitable vitamins for use in the dietary supplement include vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

The animal feed formulation may include one or more forms of an effective amount of any of the vitamins described herein or otherwise known in the art. Exemplary vitamins include vitamin K, vitamin D, vitamin C, and biotin. An “effective amount” of a vitamin typically quantifies an amount at least about 10% of the United States Recommended Daily Allowance (“RDA”) of that particular vitamin for a subject. It is contemplated, however, that amounts of certain vitamins exceeding the RDA may be beneficial for certain animals. For example, the amount of a given vitamin may exceed the applicable RDA by 100%, 200%, 300%, 400%, 500% or more.

ii. Minerals

Generally, the animal feed formulation may include one or more minerals or mineral sources. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

Generally speaking, the animal feed formulation may include one or more forms of an effective amount of any of the minerals described herein or otherwise known in the art. An “effective amount” of a mineral typically quantifies an amount at least about 10% of the United States Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 100%, 200%, 300%, 400%, 500% or more. Typically, the amount of mineral included in the dietary supplement may range from about 1 mg to about 1500 mg, about 5 mg to about 500 mg, or from about 50 mg to about 500 mg per dosage.

iii. Essential Fatty Acids

Optionally, the animal feed formulation may include a source of an essential fatty acid. The essential fatty acid may be isolated or it may be an oil source or fat source that contains an essential fatty acid. In one embodiment, the essential fatty acid may be a polyunsaturated fatty acid (PUFA), which has at least two carbon-carbon double bonds generally in the cis-configuration. The PUFA may be a long chain fatty acid having at least 18 carbons atoms. The PUFA may be an omega-3 fatty acid in which the first double bond occurs in the third carbon-carbon bond from the methyl end of the carbon chain (i.e., opposite the carboxyl acid group). Examples of omega-3 fatty acids include alpha-linolenic acid (18:3, ALA), stearidonic acid (18:4), eicosatetraenoic acid (20:4), eicosapentaenoic acid (20:5; EPA), docosatetraenoic acid (22:4), n-3 docosapentaenoic acid (22:5; n-3DPA), and docosahexaenoic acid (22:6; DHA). The PUFA may also be an omega-5 fatty acid, in which the first double bond occurs in the fifth carbon-carbon bond from the methyl end. Exemplary omega-5 fatty acids include myristoleic acid (14:1), myristoleic acid esters, and cetyl myristoleate. The PUFA may also be an omega-6 fatty acid, in which the first double bond occurs in the sixth carbon-carbon bond from the methyl end. Examples of omega-6 fatty acids include linoleic acid (18:2), gamma-linolenic acid (18:3), eicosadienoic acid (20:2), dihomo-gamma-linolenic acid (20:3), arachidonic acid (20:4), docosadienoic acid (22:2), adrenic acid (22:4), and n-6 docosapentaenoic acid (22:5). The fatty acid may also be an omega-9 fatty acid, such as oleic acid (18:1), eicosenoic acid (20:1), mead acid (20:3), erucic acid (22:1), and nervonic acid (24:1).

In another embodiment, the essential fatty acid source may be a seafood-derived oil. The seafood may be a vertebrate fish or a marine organism, such that the oil may be fish oil or marine oil. The long chain (20C, 22C) omega-3 and omega-6 fatty acids are found in seafood. The ratio of omega-3 to omega-6 fatty acids in seafood ranges from about 8:1 to 20:1. Seafood from which oil rich in omega-3 fatty acids may be derived include, but are not limited to, abalone scallops, albacore tuna, anchovies, catfish, clams, cod, gem fish, herring, lake trout, mackerel, menhaden, orange roughly, salmon, sardines, sea mullet, sea perch, shark, shrimp, squid, trout, and tuna.

In yet another embodiment, the essential fatty acid source may be a plant-derived oil. Plant and vegetable oils are rich in omega-6 fatty acids. Some plant-derived oils, such as flaxseed oil, are especially rich in omega-3 fatty acids. Plant or vegetable oils are generally extracted from the seeds of a plant, but may also be extracted from other parts of the plant. Plant or vegetable oils that are commonly used for cooking or flavoring include, but are not limited to, acai oil, almond oil, amaranth oil, apricot seed oil, argan oil, avocado seed oil, babassu oil, ben oil, blackcurrant seed oil, Borneo tallow nut oil, borage seed oil, buffalo gourd oil, canola oil, carob pod oil, cashew oil, castor oil, coconut oil, coriander seed oil, corn oil, cottonseed oil, evening primrose oil, false flax oil, flax seed oil, grapeseed oil, hazelnut oil, hemp seed oil, kapok seed oil, Iallemantia oil, linseed oil, macadamia oil, meadowfoam seed oil, mustard seed oil, okra seed oil, olive oil, palm oil, palm kernel oil, peanut oil, pecan oil, pequi oil, perilla seed oil, pine nut oil, pistachio oil, poppy seed oil, prune kernel oil, pumpkin seed oil, quinoa oil, ramtil oil, rice bran oil, safflower oil, sesame oil, soybean oil, sunflower oil, tea oil, thistle oil, walnut oil, or wheat germ oil. The plant-derived oil may also be hydrogenated or partially hydrogenated.

In still a further embodiment, the essential fatty acid source may be an algae-derived oil. Commercially available algae-derived oils include those from Crypthecodinium cohnii and Schizochytrium sp. Other suitable species of algae, from which oil is extracted, include Aphanizomenon flos-aquae, Bacilliarophy sp., Botryococcus braunii, Chlorophyceae sp., Dunaliella tertiolecta, Euglena gracilis, Isochrysis galbana, Nannochloropsis salina, Nannochloris sp., Neochloris oleoabundans, Phaeodactylum tricornutum, Pleurochrysis carterae, Prymnesium parvum, Scenedesmus dimorphus, Spirulina sp., and Tetraselmis chui.

iv. Amino Acids

The animal feed formulation may optionally include from one to several amino acids. Suitable amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine or their hydroxy analogs. In certain embodiments, the amino acid will be selected from the essential amino acids. An essential amino acid is generally described as one that cannot be synthesized de novo by the organism, and therefore, must be provided in the diet. By way of non-limiting example, the essential amino acids for humans include: L-histidine, L-isoleucine, L-leucine, L-lysine, L-methionine, L-phenylalanine, L-valine and L-threonine.

v. Antioxidants

The animal feed formulation may include one or more suitable antioxidants. As will be appreciated by a skilled artisan, the suitability of a given antioxidant will vary depending upon the species to which the dietary supplement will be administered. Non-limiting examples of antioxidants include ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-carotene, beta-apo-carotenoic acid, carnosol, carvacrol, catechins, cetyl gallate, chlorogenic acid, citric acid and its salts, p-coumaric acid, curcurin, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, propyl gallate, polyphosphates, quercetin, trans-resveratrol, rosmarinic acid, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, zeaxanthin, or combinations thereof.

Natural antioxidants that may be included in the dietary supplement include, but are not limited to, apple peel extract, blueberry extract, carrot juice powder, clove extract, coffee berry, coffee bean extract, cranberry extract, eucalyptus extract, ginger powder, grape seed extract, green tea, olive leaf, parsley extract, peppermint, pimento extract, pomace, pomegranate extract, rice bran extract, rosehips, rosemary extract, sage extract, tart cherry extract, tomato extract, turmeric, and wheat germ oil.

vi. Anti-Inflammatory Agents

The animal feed formulation may optionally include at least one anti-inflammatory agent. In one embodiment, the anti-inflammatory agent may be a synthetic non-steroidal anti-inflammatory drug (NSAID) such as acetylsalicylic acid, dichlophenac, indomethacin, oxamethacin, ibuprofen, indoprofen, naproxen, ketoprofen, mefamanic acid, metamizole, piroxicam, and celecoxib. In an alternate embodiment, the anti-inflammatory agent may be a prohormone that modulates inflammatory processes. Suitable prohormones having this property include prohormone convertase 1, proopiomelanocortin, prohormone B-type natriuretic peptide, SMR1 prohormone, and the like. In another embodiment, the anti-inflammatory agent may be an enzyme having anti-inflammatory effects. Examples of anti-inflammatory enzymes include bromelain, papain, serrapeptidase, and proteolytic enzymes such as pancreatin (a mixture of trypsin, amylase and lipase).

In still another embodiment, the anti-inflammatory agent may be a peptide with anti-inflammatory effects. For example, the peptide may be an inhibitor of phospholipase A2, such as antiflammin-1, a peptide that corresponds to amino acid residues 246-254 of lipocortin; antiflammin-2, a peptide that corresponds to amino acid residues 39-47 of uteroglobin; S7 peptide, which inhibits the interaction between interleukin 6 and interleukin 6 receptor; RP1, a prenyl protein inhibitor; and similar peptides. Alternatively, the anti-inflammatory peptide may be cortistatin, a cyclic neuropeptide related to somatostatin, or peptides that correspond to an N-terminal fragment of SV-IV protein, a conserved region of E-, L-, and P-selectins, and the like. Other suitable anti-inflammatory preparations include collagen hydrolysates and milk micronutrient concentrates (e.g., MicroLactin® available from Stolle Milk Biologics, Inc., Cincinnati, OH), as well as milk protein hydrolysates, casein hydrolysates, whey protein hydrolysates, and plant protein hydrolysates.

In a further embodiment, the anti-inflammatory agent may be a probiotic that has been shown to modulate inflammation. Suitable immunomodulatory probiotics include lactic acid bacteria such as acidophilli, lactobacilli, and bifidophilli. In yet another embodiment, the anti-inflammatory agent may be a plant extract having anti-inflammatory properties. Non-limiting examples of suitable plant extracts with anti-inflammatory benefits include blueberries, boswella, black catechu and Chinese skullcap, celery seed, chamomile, cherries, devils claw, eucalyptus, evening primrose, ginger, hawthorne berries, horsetail, Kalopanax pictus bark, licorice root, turmeric, white wallow, willow bark, and yucca.

vii. Herbals

The animal feed formulation may optionally include at least one herb or herbal derivative. Suitable herbals and herbal derivatives, as used herein, refer to herbal extracts, and substances derived from plants and plant parts, such as leaves, flowers, and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, skullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yarn, willow, wintergreen, witch hazel, wood betony, wormwood, yarrow, yellow dock, yerba santa, yucca and combinations thereof.

viii. Pigments

The animal feed formulation may optionally include at least one pigment. Suitable non-limiting pigments include actinioerythrin, alizarin, alloxanthin, β-apo-2′-carotenal, apo-2-lycopenal, apo-6′-lycopenal, astacein, astaxanthin, azafrinaldehyde, aacterioruberin, aixin, α-carotine, β-carotine, γ-carotine, β-carotenone, canthaxanthin, capsanthin, capsorubin, citranaxanthin, citroxanthin, crocetin, crocetinsemialdehyde, crocin, crustaxanthin, cryptocapsin, α-cryptoxanthin, β-cryptoxanthin, cryptomonaxanthin, cynthiaxanthin, decaprenoxanthin, dehydroadonirubin, diadinoxanthin, 1,4-diamino-2,3-dihydroanthraquinone, 1,4-dihydroxyanthraquinone, 2,2′-diketospirilloxanthin, eschscholtzxanthin, eschscholtzxanthone, flexixanthin, foliachrome, fucoxanthin, gazaniaxanthin, hexahydrolycopene, hopkinsiaxanthin, hydroxyspheriodenone, isofucoxanthin, loroxanthin, lutein, luteoxanthin, lycopene, lycopersene, lycoxanthin, morindone, mutatoxanthin, neochrome, neoxanthin, nonaprenoxanthin, OH-Chlorobactene, okenone, oscillaxanthin, paracentrone, pectenolone, pectenoxanthin, peridinin, phleixanthophyll, phoeniconone, phoenicopterone, phoenicoxanthin, physalien, phytofluene, pyrrhoxanthininol, quinones, rhodopin, rhodopinal, rhodopinol, rhodovibrin, rhodoxanthin, rubixanthone, saproxanthin, semi-α-carotenone, semi-β-carotenone, sintaxanthin, siphonaxanthin, siphonein, spheroidene, tangeraxanthin, torularhodin, torularhodin methyl ester, torularhodinaldehyde, torulene, 1,2,4-trihydroxyanthraquinone, triphasiaxanthin, trollichrome, vaucheriaxanthin, violaxanthin, wamingone, xanthin, zeaxanthin, α-zeacarotene, or combinations thereof.

ix. Pharmaceutical Acceptable Agents

The animal feed formulation may optionally include at least one pharmaceutical acceptable agent. Suitable non-limiting pharmaceutically acceptable agents include an acid/alkaline-labile drug, a pH dependent drug, or a drug that is a weak acid or a weak base. Examples of acid-labile drugs include statins (e.g., pravastatin, fluvastatin and atorvastatin), antiobiotics (e.g., penicillin G, ampicillin, streptomycin, erythromycin, clarithromycin and azithromycin), nucleoside analogs (e.g., dideoxyinosine (ddl or didanosine), dideoxyadenosine (ddA), dideoxycytosine (ddC), salicylates (e.g., aspirin), digoxin, bupropion, pancreatin, midazolam, and methadone. Drugs that are only soluble at acid pH include nifedipine, emonapride, nicardipine, amosulalol, noscapine, propafenone, quinine, dipyridamole, josamycin, dilevalol, labetalol, enisoprost, and metronidazole. Drugs that are weak acids include phenobarbital, phenytoin, zidovudine (AZT), salicylates (e.g., aspirin), propionic acid compounds (e.g., ibuprofen), indole derivatives (e.g., indomethacin), fenamate compounds (e.g., meclofenamic acid), pyrrolealkanoic acid compounds (e.g., tolmetin), cephalosporins (e.g., cephalothin, cephalaxin, cefazolin, cephradine, cephapirin, cefamandole, and cefoxitin), 6-fluoroquinolones, and prostaglandins. Drugs that are weak bases include adrenergic agents (e.g., ephedrine, desoxyephedrine, phenylephrine, epinephrine, salbutamol, and terbutaline), cholinergic agents (e.g., physostigmine and neostigmine), antispasmodic agents (e.g., atropine, methantheline, and papaverine), curariform agents (e.g., chlorisondamine), tranquilizers and muscle relaxants (e.g., fluphenazine, thioridazine, trifluoperazine, chlorpromazine, and triflupromazine), antidepressants (e.g., amitriptyline and nortriptyline), antihistamines (e.g., diphenhydramine, chlorpheniramine, dimenhydrinate, tripelennamine, perphenazine, chlorprophenazine, and chlorprophenpyridamine), cardioactive agents (e.g., verapamil, diltiazem, gallapomil, cinnarizine, propranolol, metoprolol and nadolol), antimalarials (e.g., chloroquine), analgesics (e.g., propoxyphene and meperidine), antifungal agents (e.g., ketoconazole and itraconazole), antimicrobial agents (e.g., cefpodoxime, proxetil, and enoxacin), caffeine, theophylline, and morphine. In another embodiment, the drug may be a biphosphonate or another drug used to treat osteoporosis. Non-limiting examples of a biphosphonate include alendronate, ibandronate, risedronate, zoledronate, pamidronate, neridronate, olpadronate, etidronate, clodronate, and tiludronate. Other suitable drugs include estrogen, selective estrogen receptor modulators (SERMs), and parathyroid hormone (PTH) drugs. In yet another embodiment, the drug may be an antibacterial agent. Suitable antibiotics include aminoglycosides (e.g., amikacin, gentamicin, kanamycin, neomycin, netilmicin, streptomycin, and tobramycin), carbecephems (e.g., loracarbef), a carbapenem (e.g., certapenem, imipenem, and meropenem), cephalosporins (e.g., cefadroxil cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, and ceftriaxone), macrolides (e.g., azithromycin, clarithromycin, dirithromycin, erythromycin, and troleandomycin), monobactam, penicillins (e.g., amoxicillin, ampicillin, carbenicillin, cloxacillin, dicloxacillin, nafcillin, oxacillin, penicillin G, penicillin V, piperacillin, and ticarcillin), polypeptides (e.g., bacitracin, colistin, and polymyxin B), quinolones (e.g., ciprofloxacin, enoxacin, gatifloxacin, levofloxacin, lomefloxacin, moxifloxacin, norfloxacin, ofloxacin, and trovafloxacin), sulfonamides (e.g., mafenide, sulfacetamide, sulfamethizole, sulfasalazine, sulfisoxazole, and trimethoprim-sulfamethoxazole), and tetracyclines (e.g., demeclocycline, doxycycline, minocycline, and oxytetracycline). In an alternate embodiment, the drug may be an antiviral protease inhibitor (e.g., amprenavir, fosamprenavir, indinavir, lopinavir/ritonavir, ritonavir, saquinavir, and nelfinavir). In still another embodiment, the drug may be a cardiovascular drug. Examples of suitable cardiovascular agents include cardiotonic agents (e.g., digitalis (digoxin), ubidecarenone, and dopamine), vasodilating agents (e.g., nitroglycerin, captopril, dihydralazine, diltiazem, and isosorbide dinitrate), antihypertensive agents (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril), beta blockers (e.g., levobunolol, pindolol, timolol maleate, bisoprolol, carvedilol, and butoxamine), alpha blockers (e.g., doxazosin, prazosin, phenoxybenzamine, phentolamine, tamsulosin, alfuzosin, and terazosin), calcium channel blockers (e.g., amlodipine, felodipine, nicardipine, nifedipine, nimodipine, nisoldipine, nitrendipine, lacidipine, lercanidipine, verapamil, gallopamil, and diltiazem), monensin, avilamycin, salinomycin, narasin, diclaserol, tylosin, bacitracin, bacitracin zinc, and anticlot agents (e.g., dipyrimadole).

x. Excipients

A variety of commonly used excipients in animal feed formulation may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agents.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In another embodiment, the excipient may be a filler. Suitable fillers include carbohydrates, inorganic compounds, and polyvinylpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth.

In another embodiment, the excipient may be an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

In another embodiment, the excipient may include a diluent. Diluents suitable for use include pharmaceutically acceptable saccharides such as sucrose, dextrose, lactose, microcrystalline cellulose, fructose, xylitol, and sorbitol; polyhydric alcohols; a starch; pre-manufactured direct compression diluents; and mixtures of any of the foregoing.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the embodiment, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in the present invention depending on the embodiment.

The excipient may include a taste-masking agent. Taste-masking materials include, e.g., cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as OpadryAMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking materials contemplated are those described in U.S. Pat. Nos. 4,851,226; 5,075,114; and 5,876,759, each of which is hereby incorporated by reference in its entirety.

In various embodiments, the excipient may include a pH modifier. In certain embodiments, the pH modifier may include sodium carbonate or sodium bicarbonate.

The amount and types of ingredients and other excipients useful in the animal feed formulation are described further in the examples.

### II. Feed Additive Composition

One aspect of the present disclosure encompasses feed additive compositions for non-human animals comprising a glycine compound, such as DMG. Other optional additives may be further included. The feed additive composition may be added to a basal animal diet for administration to the non-human animals. The feed additive composition may be formulated with basal animal diets to prepare the feed compositions described in Section I.

In some embodiments, DMG in a basal animal diet comprises about 65% to about 85% DMG, preferably about 70% to about 80% DMG, and more preferably about 75% to about 79% DMG.

In various embodiments, a feed additive composition may be introduced to a basal animal diet by way of various methods, depending on whether the feed additive composition is in a liquid or solid form. Non-limiting examples of introducing the feed additive composition to a basal animal diet may be formulating the feed additive composition into the basal animal diet, top-dressing the solid composition of a basal animal diet, spraying a liquid feed additive composition onto a basal animal diet, or combinations thereof. It will be recognized that, when the feed additive is introduced to a basal animal diet, the amount of the feed additive introduced to a basal animal diet is sufficient to provide the therapeutically effective amount of the DMG extract in the diet of the animal.

In addition to DMG, a feed additive composition may further comprise at least one additional ingredient such as vitamins, minerals, amino acids, antioxidants, probiotics, essential fatty acids, and pharmaceutically acceptable excipients. Such ingredients may be as described in Section I(b) above.

### III. Methods of Using

Another aspect of the disclosure encompasses methods of using a feed composition. The methods comprise administering the animal feed composition to non-human animals, in particular pigs, for example, sows, boars, or post weaning pigs. Preferably, a feed composition is administered orally to non-human animals. A feed composition may be as described in Section (I).

The timing and duration of administration of a composition of the invention to an animal can and will vary. The feed composition may be administered throughout the period of feeding the animal. Alternatively, the feed composition may be administered at specific periods during the growth and development of the animal, for instance, during periods of lactation. A composition may also be administered at various intervals. For instance, a composition may be administered daily, weekly, monthly, or over a number of months. In some embodiments, a composition is administered weekly. In other embodiments, a composition is administered monthly. In preferred embodiments, a composition is administered daily. As it will be recognized in the art, the duration of treatment can and will vary depending on the growth and health of the animal.

The present disclosure is directed to a method for improving carcass characteristics in a non-human animal. The method comprises feeding the non-human animal the feed composition described in Section I.

The present disclosure is directed to a method for improving milk composition in a non-human animal. The milk composition comprises milk fat content, milk energy concentration, and/or milk protein content. The method comprises feeding the non-human animal the feed composition described in Section I.

The present disclosure is directed to a method for increasing protein deposition and reducing protein turnover in a non-human animal. The method comprises feeding the non-human animal the feed composition described in Section I.

The present disclosure is further directed to a method for improving milk production in a non-human animal. The method comprises feeding the non-human animal the feed composition described in Section I.

The present disclosure is directed to a method for improving growth performance in a non-human animal in a non-human animal. The method comprises feeding the non-human animal the feed composition described in Section I.

The present disclosure is directed to a method for improving livability in a non-human animal in a non-human animal. The method comprises feeding the non-human animal the feed composition described in Section I.

The present disclosure is directed to a method for reducing morbidity in a non-human animal. The method comprises feeding the non-human animal the feed composition described in Section I.

The present disclosure is directed to a method for reducing greenhouse gas emissions and carbon footprint. The method comprises feeding the non-human animal the feed composition described in Section I.

### Definitions

Unless otherwise defined, all technical and scientific terms used herein have the same meaning as is commonly understood by one of skill in the art to which this disclosure belongs at the time of filing. If specifically defined, then the definition provided herein takes precedent over any dictionary or extrinsic definition. Further, unless otherwise required by context, singular terms shall include pluralities, and plural terms shall include the singular. Herein, the use of “or” means “and/or” unless stated otherwise. All patents and publications referred to herein are incorporated by reference.

When introducing elements of the present disclosure or the embodiments(s) thereof, the articles “a,” “an,” “the,” and “said” are intended to mean that there are one or more of the elements. The terms “comprising,” “including,” and “having” are intended to be inclusive and mean that there may be additional elements other than the listed elements.

Although the disclosure described herein is susceptible to various modifications and alternative iterations, specific embodiments thereof have been described in greater detail above. It should be understood, however, that the detailed description is not intended to limit the disclosure to the specific embodiments disclosed. Rather, it should be understood that the disclosure is intended to cover all modifications, equivalents, and alternatives falling within the spirit and scope of the disclosure as defined by the claim language.

### Examples

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Carcass Characteristics

A study was conducted to evaluate the impact of increasing dietary levels of 78% Na-DMG on carcass characteristics of commercial finishing pigs (PIC 800×PIC 1050). A total of 723 mixed-sex finishing pigs (initial BW=179.5±1.0 lb) were housed in pens with 25 to 27 pigs per pen. Pens within block were assigned randomly to one of four dietary treatments containing 0 (Control), 0.25 (DMG_0.25), 0.50 (DMG_0.5) or 1.0 (DMG_1.0) lb DMG/ton of feed with 7 replicates per treatment. This experiment extended over a 6-week period until pigs were harvested at 292.5±1.7 lb of BW.

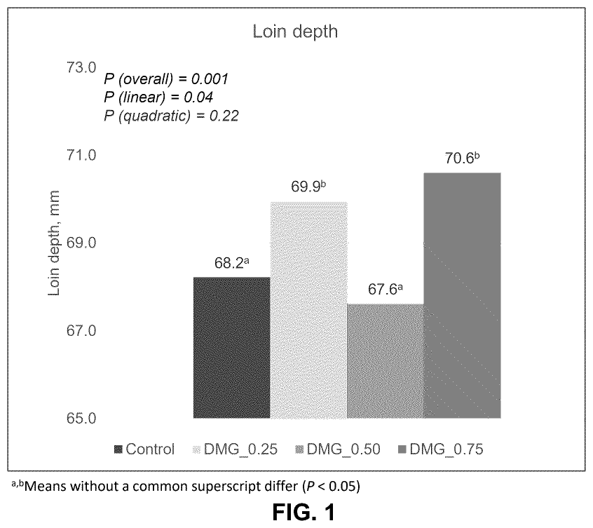

Loin depth was linearly improved as dietary DMG level increased (P=0.04; FIG. 1). Specifically, feeding DMG at 0.25 and 0.75 lb/ton significantly (P<0.05) improved loin depth by 2.5% and 3.5%, respectively, compared to carcasses from pigs fed Control diet.

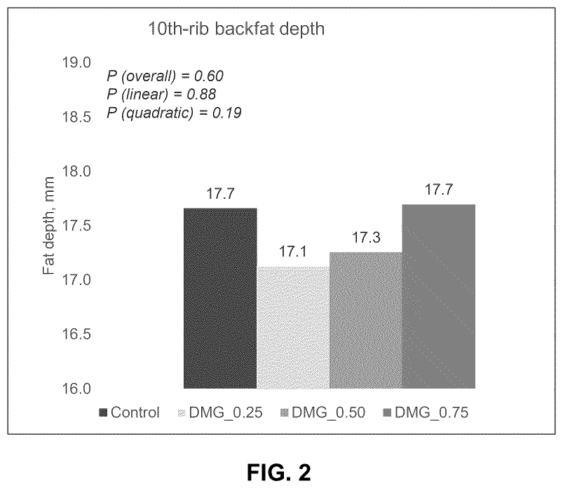

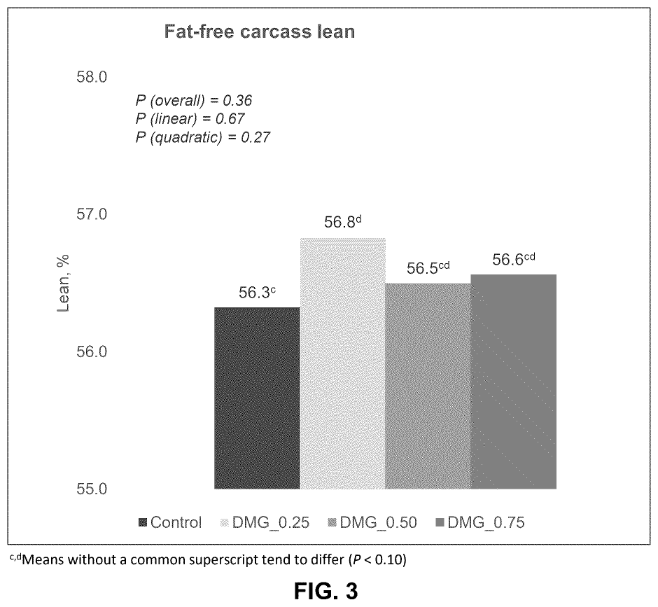

10^{th}-rib backfat depth was numerically reduced by 3.4% and 2.3% in carcasses from pigs fed DMG at 0.25 and 0.50 lb/ton, respectively (FIG. 2). Moreover, fat-free carcass lean tended to be improved when DMG was supplemented at 0.25 lb/ton (P<0.10; FIG. 3). Results from this study suggested supplementation of DMG to finishings pigs improved lean muscle deposition of the carcasses.

The increased loin depth, reduced backfat thickness, and improvements in fat-free lean would all point towards the pigs fed DMG having greater protein accretion rates and lower fat deposition rates than pigs not fed DMG. The targeted time frame would suggest that when the pig is in a protein dependent phase of growth, supplementing DMG in the diet allows the pig to shift tissue deposition rates and creates a corresponding F/G benefit as it requires more dietary energy intake for lipid accretion than protein accretion. Early in the grow out period, a pig is in an energy dependent phase of growth and not consuming high enough energy intake to shift to protein accretion rates and which may explain any lack of response to DMG in that stage of production.

### Example 2. Improving Milk Quality

A total of 77 sows (PIC 1050) were randomly assigned one of three dietary treatments: (1) a control diet; (2) diet containing Na-DMG (78%) at 0.5 lb/ton fed from day 110 of gestation through lactation; (3) diet containing Na-DMG (78%) at 0.5 lb/ton fed from farrowing through lactation. Total milk fat content measured on day 10 to 14 of lactation was improved by 10.9% in sows fed DMG from day 110 of gestation through lactation and improved by 8.3% in sows fed DMG from farrowing through lactation (Table 1).

TABLE 1
Milk fat content in sows fed Na-DMG (78%)

| | Total milk fat, % | |
|---|---|---|
| Treatment | (Day 10-14 in lactation) | |
| 1 - Control | 5.515 | |
| 2 - DMG fed at 0.5 lb/ton from day 100 | 6.114 | (+10.9%) |
| of gestation through lactation | | |
| 3 - DMG fed at 0.5 lb/ton in from | 5.973 | (+8.3%) |
| farrowing through lactation | | |

Results from this study suggested that DMG emerges as a novel and promising method to elevate milk fat content and enhance overall milk quality in lactating animals. By supplementing sow diets with DMG, significant improvements in milk fat content were observed, indicating its ability to positively modulate the composition of sow milk. This finding holds profound implications for both sow and offspring health, as milk fat content is crucial for ensuring optimal growth and development of nursing piglets. Moreover, enhanced milk quality resulting from DMG supplementation may translate into improved nutrient provision and health outcomes for newborn piglets, thereby contributing to the overall welfare and productivity of swine herds.

### Example 3. Growth Performance, Livability, and Carcass Characteristics

To evaluate the impact of increasing dietary levels of 78% sodium dimethylglycine (DMG) on growth performance, livability, and carcass characteristics of commercial finishing pigs, a total of total of 1,115 mixed-sex finishing pigs (initial BW=70.1±1.8 kg) were housed in pens with 25 to 27 pigs per pen. Pens within block were assigned randomly to one of four dietary treatments containing 0 (Control), 0.25 (DMG_0.25), 0.5 (DMG_0.5) or 1.0 (DMG_1.0) g DMG/kg of feed with 11 replicates per treatment. This experiment extended over an 8-wk period until pigs were harvested at 125.4±0.8 kg of BW.

Data were analyzed using the MIXED procedure of SAS, and preplanned orthogonal contrasts were conducted to evaluate pairwise comparisons as well as linear and quadratic effects related to the increased levels of DMG. While no linear effect was observed, there was a tendency for a quadratic response in closeout feed efficiency (G/F, P=0.08) and the percentage of pigs with morbidity (P=0.06), with the most favorable responses found in pigs fed DMG_0.25 (Table 2).

TABLE 2
Effects of feeding increasing levels of dimethylglycine (DMG) on growth performance,
livability, and carcass characteristics of commercial finishing pigs.

| | | | | | | P-values | | |
|---|---|---|---|---|---|---|---|---|
| | | | | | | | | Control |
| | Treatment | | | | | | | vs. |
| Item | Control | DMG_0.25 | DMG_0.5 | DMG_1.0 | PSE | Linear | Quadratic | DMG_0.25 |
| Growth performance | | | | | | | | |
| Start BW,¹ kg | 69.8 | 70.7 | 69.7 | 70.1 | 1.8 | 0.92 | 0.58 | 0.13 |
| End BW, kg | 124.7 | 126.4 | 124.9 | 125.8 | 0.8 | 0.45 | 0.53 | 0.05 |
| Closeout ADG, kg/day | 0.95^{x} | 1.01^{y} | 0.95^{x} | 0.97^{xy} | 0.02 | 0.75 | 0.16 | 0.02 |
| ADFI, kg/day | 2.96^{ab} | 3.00^{b} | 2.93^{a} | 2.99^{ab} | 0.02 | 0.86 | 0.61 | 0.12 |
| Closeout G/F | 0.324 | 0.337 | 0.326 | 0.323 | 0.006 | 0.53 | 0.08 | 0.05 |
| Livability | | | | | | | | |
| Mortality, % | 2.1^{d} | 0.4 | 2.5^{d} | 1.4^{cd} | 0.7 | 0.99 | 0.69 | 0.08 |
| Morbidity, % | 1.1^{d} | 0.0^{c} | 0.7^{cd} | 2.2^{d} | 0.7 | 0.21 | 0.06 | 0.09 |
| Non-selected pigs | 9.2 | 8.7 | 9.4 | 6.5 | 1.6 | 0.25 | 0.49 | 0.74 |
| (lights and culls), % | | | | | | | | |
| Pigs sold to primary | 87.6 | 90.9 | 87.4 | 90.0 | 1.5 | 0.57 | 0.70 | 0.10 |
| market, % | | | | | | | | |
| Carcass characteristics | | | | | | | | |
| % of carcasses measured | 70.4 | 70.2 | 66.7 | 70.8 | | | | |
| Hot carcass weight, kg | 93.3 | 93.5 | 94.1 | 94.2 | 0.9 | 0.42 | 0.89 | 0.81 |
| Carcass yield, % | 73.2 | 72.6 | 73.6 | 73.8 | 0.7 | 0.34 | 0.52 | 0.41 |
| Fat depth, mm | 16.8 | 16.6 | 17.0 | 16.8 | 0.3 | 0.81 | 0.96 | 0.60 |
| Loin depth, mm | 63.1 | 62.5 | 62.2 | 61.7 | 0.9 | 0.28 | 0.96 | 0.71 |
| Fat-free carcass lean, % | 56.2 | 56.2 | 56.0 | 56.0 | 0.2 | 0.43 | 0.95 | 0.81 |

^{a,b}Means without a common superscript within a row differ (P < 0.05)
^{x,y}Means without a common superscript within a row tend to differ (P < 0.10)
¹Start BW was used as covariate in all analyses
BW = body weight;
ADG = average daily gain;
ADFI = average daily feed intake;
G/F = gain/feed

Pairwise comparisons between Control and DMG_0.25 treatments suggest that pigs receiving DMG_0.25 showed a 6% increase in closeout ADG (1.01 vs. 0.95 kg/day; P=0.02) and a 4% increase in closeout G/F (0.337 vs. 0.324; P=0.05) compared with those fed Control diet. Furthermore, DMG supplemented at 0.25 g/kg tended to be associated with lower mortality (P=0.08), lower morbidity (P=0.09) and more pigs sold to the primary market (P=0.10) compared with Control. In contrast, dietary supplementation of higher concentrations of DMG at 0.5 and 1.0 g/kg were not different compared with Control (P>0.10).

Carcass characteristics, including hot carcass weight, carcass yield, fat depth, loin depth, and fat-free carcass lean, were not different among treatments. The findings of this study suggest dietary supplementation of DMG at 0.25 g/kg of feed enhanced growth performance and livability in finishing pigs. However, feeding higher concentrations of DMG, up to 1.0 g/kg of feed, did not produce different outcomes compared with Control. These results indicate the unexpected benefits of DMG at specific inclusion rate in optimizing the production and health of finishing pigs.

### Example 4

A total of 1,180 growing pigs (initial BW=51.4±1.0 lb) were housed in pens with 25 to 27 pigs per pen. Pens within block were assigned randomly to one of four dietary treatments containing 0 (Control), 0.25 (DMG_0.25), 0.50 (DMG_0.5) or 1.0 (DMG_1.0) lb DMG/ton of feed with 11 replicates per treatment. This experiment extended over a 17-wk period until pigs were harvested at 292.5±1.7 lb of BW. The data is presented in Table 3.

Signs of lameness were recorded, and percentage of pigs removed to hospital pens due to lameness were calculated (Table 3). Feeding DMG to pigs, regardless of dietary concentration, either statistically (P<0.10) or numerically (P>0.10) reduced incidence of lameness compared to Control.

TABLE 3
Incidence of lameness in finishings pigs fed DMG

| | Control | DMG_0.25 | DMG_0.50 | DMG_0.75 |
|---|---|---|---|---|
| Number of | 297 | 293 | 295 | 295 |
| pigs started | | | | |
| Lameness, % | 2.0%^{c} | 0.3%^{d} | 0.7%^{cd} | 1.4%^{cd} |

^{c,d}Means without a common superscript tend to differ (P < 0.10)

### Example 5: Reduces Overall Morbidity and Mortality in Finishing Pigs

Three studies were compiled to evaluate the effects of dietary supplementation of Na-DMG (78%) on morbidity and mortality in finishing pigs. In each study, pigs were fed either a Control diet or diet containing Na-DMG at 0.50 lb/ton from approximately 180 lb of body weight until harvested at 290 lb for a period of 6 to 8 weeks. Feeding DMG significantly reduced morbidity by 72% and total removal by 53% compared to pigs fed Control diet (Table 4). Reasons of morbidity and mortality included, but not limited to, lameness, ileitis, bacterial infection, injury, prolapse, intestinal torsion, stomach ulcers, abdominal rupture, bloat, failure to eat. The lower morbidity and mortality rate in DMG group also led to more pigs sold to primary market (93.1 vs. 91.1%).

TABLE 4
Effects of dietary supplementation of Na-DMG (78%)
on morbidity and mortality in finishing pigs.

| | Meta-analysis (3-trial combined) | | | |
|---|---|---|---|---|
| Item | Control | DMG_0.50 | PSE | P-value |
| # of Pens | 33 | 44 | | |
| # of Pigs | 847 | 1113 | | |
| Start BW, lb | 179.5 | 180.5 | 2.24 | 0.55 |
| Growth Performance | | | | |
| Mortality, % | 1.77 | 1.08 | N/A | 0.20 |
| Morbidity, % | 1.30 | 0.36 | N/A | 0.02 |
| Total removal, % | 3.07 | 1.44 | N/A | 0.01 |
| Total pigs finished, % | 96.9 | 98.6 | 0.57 | 0.04 |
| Non-selected | 5.79 | 5.48 | N/A | 0.78 |
| (culls and lights), % | | | | |
| Pigs sold to primary market, % | 91.1 | 93.1 | 0.88 | 0.12 |

### Example 6: Changes on NEFA

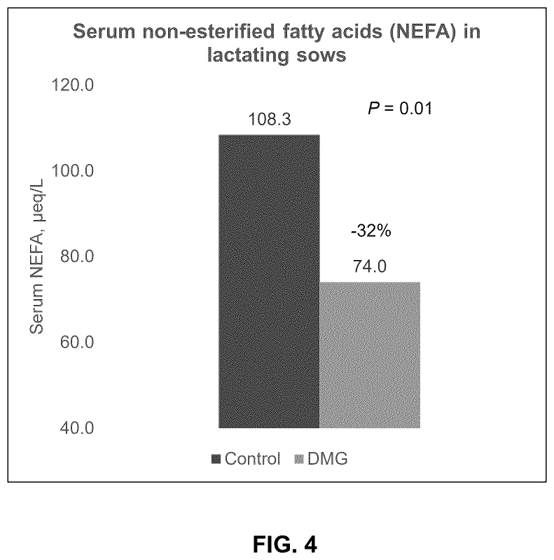

Serum non-esterified fatty acids (NEFA) are molecules released from adipose tissue during periods of energy demand or catabolic status. In research of energy metabolism, NEFA level has been used as biomarker to assess balance between fat mobilization and energy expenditure. In lactating cows, lower blood NEFA levels indicate improved metabolic status and energy balance which suggest greater milk production and better body condition. In our research, serum NEFA measured at weaning was significantly reduced by 32% in lactating sows fed 445 mg Na-DMG per day compared to sows fed Control diet (FIG. 4). This finding suggests that DMG may positively impact energy balance and improve energy availability during periods of increased energy demand, thereby facilitating energy-dependent processes such as lean tissue growth or milk production.

### Example 7: Reduces Greenhouse Gas Emissions

Animals produce greenhouse gases due to biological processes for growth and maintenance functions. Pig production would therefore be a source of greenhouse gases and wean to finish production would result in production of carbon dioxide, methane, and nitrous oxide emissions. Using the Iowa Pork Industry's sustainability calculator, we calculated the impact feedings of DMG in late finishing would have on greenhouse gas emissions. The table below details the F/G and mortality benefit that was observed in three studies directed to supplying DMG in late finishing. We then took similar estimates for early performance and estimated F/G and mortality for an entire grow-out period to use in the calculator. The calculator provides equivalent lb. of carbon dioxide per lb. or market weight for comparison purposes. By adding DMG in late finishing, carbon dioxide emissions were reduced by 3% during the entire wean to finish grow-out. This finding would suggest that DMG can reduce greenhouse emissions and improve the sustainability of pork production.

| | Meta-analysis (3-trial combined) | | | |
|---|---|---|---|---|
| | | DMG @ 0.5 | | |
| Item | Control | lb/ton | % Change | P-value |
| # of Pens | 33 | 44 | | |
| # of Pigs | 847 | 1113 | | |
| Late Finishing | | | | |
| Testing Period | | | | |
| Closeout F/G | 3.46 | 3.30 | −4.6% | 0.006 |
| Mortality, % | 1.77 | 1.08 | −39.0% | 0.20 |
| Calculated Wean-to-Finish | | | | |
| Performance | | | | |
| Closeout F/G¹ | 2.65 | 2.58 | −2.6% | N/A |
| Mortality, %² | 3.77 | 3.08 | −18.3% | N/A |
| Carbon Dioxide lb per | 4.27 | 4.14 | −3.0% | N/A |
| lb market hog³ | | | | |

¹A similar feed budget of 353 lb per pig was combined with the late finishing feed conversion to estimate the wean-to-finish performance.
²A similar early mortality rate of 2% was combined with the late finishing mortality rate to estimate the wean-to-finish mortality.
³Estimated greenhouse gas emissions (combines methane and nitrous oxide into carbon dioxide equivalents) calculated by the Wean-to-Finish Pork Sustainability Calculator from the Iowa Pork Industry Center (https://www.extension.iastate.edu/news/new-sustainability-tool-calculates-environmental-impact-pork-production-practices).

What is claimed is:

1. A feed composition for improving carcass characteristic in a non-human animal, the composition comprising a basal animal diet supplemented with a glycine compound, wherein the carcass characteristic comprises measurements of muscle and fat deposition.

2. The feed composition of claim 1, wherein the measurements of muscle and fat deposition comprise loin depth, loin eye area, fat depth, carcass yield, and/or lean percentage.

3. The feed composition of claim 1, wherein the glycine compound is present in the feed composition at a concentration of up to about 2 lb/ton.

4. The feed composition of claim 3, wherein the glycine compound is present in the feed composition at a concentration of about 0.25 lb/ton to about 0.50 lb/ton.

5. The feed composition of claim 1, wherein the glycine compound comprises N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N,N-di-tert-butylglycine, trimethylglycine, or mixtures or salts or esters thereof.

6. The feed composition of claim 5, wherein the glycine compound is DMG.

7. The feed composition of claim 1, wherein the animal is a non-ruminant animal.

8. The feed composition of claim 1, wherein the animal is a pig.

9. The feed composition of claim 1, wherein the animal is a finishing pig.

10. A feed composition for improving milk composition in a non-human animal, the composition comprising a basal animal diet supplemented with a glycine compound; wherein the milk composition comprises milk fat content, milk energy concentration, and/or milk protein content.

11. The feed composition of claim 10, wherein the glycine compound is present in the feed composition at a concentration of up to about 2 lb/ton.

12. The feed composition of claim 11, wherein the glycine compound is present in the feed composition at a concentration of about 0.25 lb/ton to about 0.50 lb/ton.

13. The feed composition of claim 10, wherein the glycine compound comprises N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, or trimethylglycine, or mixtures or salts or esters thereof.

14. The feed composition of claim 13, wherein the glycine compound is DMG.

15. The feed composition of claim 10, wherein the animal is a non-ruminant animal.

16. The feed composition of claim 10, wherein the animal is a pig.

17. The feed composition of claim 10, wherein the animal is a sow.

18. A feed composition for increasing protein deposition and reducing protein turnover in a non-human animal, the composition comprising a basal animal diet supplemented with a glycine compound.

19. The feed composition of claim 18, wherein the glycine compound is present in the feed composition at a concentration of up to about 2 lb/ton.

20. The feed composition of claim 19, wherein the glycine compound is present in the feed composition at a concentration of about 0.25 lb/ton to about 0.50 lb/ton.

21. The feed composition of claim 18, wherein the glycine compound comprises N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, or trimethylglycine, or mixtures or salts or esters thereof.

22. The feed composition of claim 18, wherein the glycine compound is DMG.

23. The feed composition of claim 18, wherein the animal is a non-ruminant animal.

24. The feed composition of claim 18, wherein the animal is a pig.

25. The feed composition of claim 18, wherein the animal is a sow.

26. A feed composition for improving milk production in a non-human animal, the composition comprising a basal animal diet supplemented with a glycine compound.

27. The feed composition of claim 26, wherein the glycine compound is present in the feed composition at a concentration of up to about 2 lb/ton.

28. The feed composition of claim 27, wherein the glycine compound is present in the feed composition at a concentration of about 0.25 lb/ton to about 0.50 lb/ton.

29. The feed composition of claim 26, wherein the glycine compound comprises N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, or trimethylglycine, or mixtures or salts or esters thereof.

30. The feed composition of claim 26, wherein the glycine compound is DMG.

31. The feed composition of claim 26, wherein the animal is a non-ruminant animal.

32. The feed composition of claim 26, wherein the animal is a pig.

33. The feed composition of claim 26, wherein the animal is a sow.

34. A feed composition for improving growth performance in a non-human animal, the composition comprising a basal animal diet supplemented with DMG.

35. The feed composition of claim 34, wherein the glycine compound is present in the feed composition at a concentration of up to about 2 lb/ton.

36. The feed composition of claim 35, wherein the glycine compound is present in the feed composition at a concentration of about 0.25 lb/ton to about 0.50 lb/ton.

37. The feed composition of claim 34, wherein the glycine compound comprises N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, trimethylglycine, or mixtures or salts or esters thereof.

38. The feed composition of claim 34, wherein the glycine compound is DMG.

39. The feed composition of claim 34, wherein the animal is a non-ruminant animal.

40. The feed composition of claim 34, wherein the animal is a pig.

41. The feed composition of claim 34, wherein the animal is a finishing pig.

42. A feed composition for improving livability in a non-human animal, the composition comprising a basal animal diet supplemented with a glycine compound.

43. The feed composition of claim 42, wherein the glycine compound is present in the feed composition at a concentration of up to about 2 lb/ton.

44. The feed composition of claim 43, wherein the glycine compound is present in the feed composition at a concentration of about 0.25 lb/ton to about 0.50 lb/ton.

45. The feed composition of claim 42, wherein the glycine compound comprises N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, trimethylglycine, or mixtures or salts or esters thereof.

46. The feed composition of claim 45, wherein the glycine compound is DMG.

47. The feed composition of claim 42, wherein the animal is a non-ruminant animal.

48. The feed composition of claim 42, wherein the animal is a pig.

49. The feed composition of claim 42, wherein the animal is a sow.

50. The feed composition of claim 42, wherein the animal is a finishing pig.

51. A feed composition for reducing morbidity, in a non-human animal, the composition comprising a basal animal diet supplemented with a glycine compound.

52. The feed composition of claim 51, wherein the glycine compound is present in the feed composition at a concentration of up to about 2 lb/ton.

53. The feed composition of claim 52, wherein the glycine compound is present in the feed composition at a concentration of about 0.25 lb/ton to about 0.50 lb/ton.

54. The feed composition of claim 51, wherein the glycine compound comprises N,N-dimethylglycine (DMG), N,N-diethylglycine, N,N-diethylglycine, N,N-diethanolglycine, N,N-di-n-propylglycine, N,N-diisopropylglycine, N,N-di-n-butylglycine, N,N-diisobutylglycine, N₁N-di-tert-butylglycine, trimethylglycine, or mixtures or salts or esters thereof.

55. The feed composition of claim 54, wherein the glycine compound is DMG.

56. The feed composition of claim 51, wherein the animal is a non-ruminant animal.

57. The feed composition of claim 51, wherein the animal is a pig.

58. The feed composition of claim 51, wherein the animal is a sow.

59. The feed composition of claim 51, wherein the animal is a growing pig.

60. A feed composition for reducing greenhouse gas emissions and carbon footprint in a non-human animal, the composition comprising a basal animal diet supplemented with a glycine compound.

61. A feed composition for reducing lameness in a non-human animal, the composition comprising a basal animal diet supplemented with a glycine compound.

62. A method for improving carcass characteristics in a non-human animal, the method comprising: feeding the non-human animal a feed composition of claim 1.

63. A method for improving milk composition in a non-human animal, the method comprising: feeding the non-human animal a feed composition of claim 10.

64. A method for increasing protein deposition and reducing protein turnover in a non-human animal, the method comprising: feeding the non-human animal a feed composition of claim 18.

65. A method for improving milk production in a non-human animal, the method comprising: feeding the non-human animal a feed composition of claim 26.

66. A method for improving growth performance in a non-human animal, the method comprising: feeding the non-human animal a feed composition of claim 34.

67. A method for improving livability in a non-human animal, the method comprising: feeding the non-human animal a feed composition of claim 42.

68. A method for reducing morbidity, in a non-human animal, the method comprising: feeding the non-human animal a feed composition of claim 51.

69. A method for reducing lameness in a non-human animal, the method comprising: feeding the non-human animal a feed composition of claim 61.
